SEQUENCE LISTING (6 sequences)

SEQ ID NO: 1 - DNA, 21 bp, Artificial
  Synthetic Oligo for transfection experiments
acgtcgctgccatcctcgctg

SEQ ID NO: 2 - DNA, 36 bp, Artificial
  Synthetic NOTCH1 DOMAIN (FORWARD STRAND)
cccgccctgcccacgtcgctgccatcctcgctggtc

SEQ ID NO: 3 - DNA, 36 bp, Artificial
  Synthetic NOTCH1 DOMAIN (REVERSE STRAND)
gggcgggacgggtgcagcgacggtaggagcgaccag

SEQ ID NO: 4 - DNA, 9 bp, Artificial
  Synthetic NOTCH1 DOMAIN (FORWARD STRAND)
gcagcccag

SEQ ID NO: 5 - DNA, 9 bp, Artificial
  Synthetic NOTCH1 DOMAIN (REVERSE STRAND)
cgtcgggtc

SEQ ID NO: 6 - DNA, 40 bp, Artificial
  Synthetic NOTCH1 PEST
  misc_feature (16)..(19): A, T, C, G, no nucleotide
cccgccctgcccacgnnnntcgctgccatcctcgctggtc

(19) United States
(12) Patent Application Publication
(10) Pub. No.: US 2025/0332150 A1
(43) Pub. Date: Oct. 30, 2025

(54) METHODS FOR TREATING NOTCH1-DRIVEN CANCERS

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE (CNRS), Paris (FR); ASSISTANCE PUBLIQUE-HÔPITAUX DE PARIS (APHP), Paris (FR); UNIVERSITÉ PARIS CITÉ, Paris (FR)

(72) Inventors: Vahid ASNAFI, Paris (FR); Mathieu SIMONIN, Paris (FR); Olivier HERMINE, Paris (FR)

(21) Appl. No.: 18/708,645

(22) PCT Filed: Dec. 1, 2021
(86) PCT No.: PCT/IB2021/000837

§ 371 Date: May 9, 2024

(51) Int. Cl. A61K 31/439; A61K 31/704; A61K 31/7048; A61P 35/02; G01N 33/68

(52) U.S. Cl. CPC A61K 31/439; A61K 31/704; A61K 31/7048; A61P 35/02; G01N 33/6872; G01N 2800/52

(57) ABSTRACT

T-cell acute lymphoblastic leukemias (T-ALL) are aggressive hematological malignancies associated with poor clinical outcome. TP53 alterations (TP53^{Alt}) were rarely identified in T-ALL at diagnosis and their prognostic impact remains unclear. In a cohort of 476 adults and pediatric T-ALL, TP53^{Alt} were observed in 4% of cases and were associated with chemoresistance and poor prognosis. APR-246, a small compound which restores wild-type configuration to mutated p53, showed efficacy in T-ALL harboring TP53 mutations. More importantly, in TP53 germline T-ALL, Notch 1 pathway gain of function mutations were associated with substantial sensitivity to APR-246. Mechanistically, Notch 1 activation via p53 downregulation and subsequent ferroptosis induction led to preferential APR-246 sensitivity. Given that Notch 1 pathway oncogenic activation is present in more than 70% of T-ALLs, these observations pave the way for promising perspectives in T-ALL treatment which could benefit from the Achilles heel associated with Notch 1 activation sensitizing leukemia cells to APR-246-induced ferroptosis, thus extending the use of APR-246 in T-ALL beyond TP53 alterations.

### FIELD OF THE INVENTION

The present invention is in the field of medicine, in particular oncology.

### BACKGROUND OF THE INVENTION

Among hematological malignancies, T-cell acute lymphoblastic leukemia (T-ALL) represents a class of aggressive tumors with dismal clinical outcome. While the intensification of multi-agent chemotherapy protocols has dramatically improved prognosis, refractory and relapsed cases are clinically challenging due to limited therapeutic options (1, 2). p53 is a transcription factor and a master tumor suppressor gene frequently altered in cancer (3). In contrast to carcinomas and other hematological malignancies, TP53 alterations (TP53^{Alt}), encompassing mutations (TP53^{Mut}) and/or pan-exon deletions (TP53^{Del}), are remarkably rare at diagnosis in T-ALL, and their clinical implication remains elusive (4, 5). Critically, TP53^{Alt} have been reported to be acquired in up to 20% of the relapsed T-ALL cases, when they convey a deleterious prognosis (6-8). A seducing hypothesis to explain the scarcity of TP53^{Alt} in T-ALL stems from the intricate cross-regulation of the p53, CDKN2A/ARF and Notch1 pathways. Most T-ALLs harbor genetic alterations of NOTCH1, leading to constitutive Notch1 signaling (9). Several studies showed that Notch1 regulates the p53 pathway (10). The Notch1 intracellular domain (ICN1) can complex with p53 to block its transactivation (11). CDKN2A/ARF binds to MDM2, the E3 ubiquitin ligase of p53, and disrupts the interaction between MDM2 and p53, preventing p53 ubiquitination and degradation. The CDKN2A locus, which encodes the p53 regulator ARF, is frequently deleted in T-ALL. Notch1 also downregulates ARF (12, 13). Notch1-mediated activation of PI3K/Akt signaling increases MDM2 activity and reduces p53 function (14). PTEN, a negative regulator of PI3K/Akt signaling, is inactivated in 15% of T-ALL leading to Akt activation and subsequently MDM2 upregulation resulting in p53 degradation (14, 15). As such, the main genetic lesions in T-ALL all downregulate p53 function, even in the absence of TP53 alterations. Strong evidence supporting the hypothesis that Notch1 induced down tuning of p53 drives T-ALL (10) is the observation that p53 stabilization using Nutlin-3 or ionizing radiation is sufficient for tumor regression in a Notch1-driven context (13, 16).

These observations position the interplay between the p53 and Notch1 pathways as a potential target to treat T-ALL. Indeed, the restoration of wild-type p53 function in a Notch1-altered context could represent an elegant strategy to counter T-ALL progression. APR-246, a small molecule targeting p53, has shown impressive results in TP53^{Mut} cancers. It interacts with mutated p53 to restore its wild-type (WT) conformation and function (17, 18). APR-246 also exerts a p53-independent function on the reactive oxygen species (ROS) balance, by increasing ROS through depletion of the glutathione (GSH) pool, thus inducing ferroptosis, a regulated cell death program triggered in the event of oxidative stress response failure (19, 20).

### SUMMARY OF THE INVENTION

The present invention is defined by the claims. In particular, the present invention relates to a method of treating a NOTCH1-driven cancer in a subject in need thereof comprising administering a ferroptosis inducer and to a method for predicting the response of a subject suffering from a NOTCH1-driven cancer to a ferroptosis inducer wherein a NOTCH1 pathway activation indicates that the subject is responder to the ferroptosis inducer.

### DETAILED DESCRIPTION OF THE INVENTION

Here, the Inventors produce the first comprehensive analysis of TP53^{Alt} and the associated oncogenetic landscape in an extensive cohort of 476 patients newly diagnosed with T-ALL. TP53^{Alt} were observed in 4% of T-ALL at diagnosis and were associated with poor prognosis. They provide a basis for targeting p53-mutated T-ALL using the novel small molecule APR-246. Critically, they demonstrate that APR-246 also has a clear efficacy on TP53^{WT} NOTCH^{Mut} T-ALL, thus identifying p53-unrelated functions in T-ALL.

In a first aspect, the present invention relates to a method of treating a NOTCH1-driven cancer in a subject in need thereof comprising administering a ferroptosis inducer.

As used herein, the term “subject” or “patient” denotes a mammal, preferably a human. Typically, a subject according to the invention refers to any subject afflicted with or susceptible to be afflicted with a NOTCH1-driven cancer, in particular a T-cell acute lymphoblastic leukemia.

As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: leukemia; lymphoid leukemia; T-cell acute lymphoblastic leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease. In some embodiments, the cancer is a T-cell acute lymphoblastic leukemia.

As used herein, the term “NOTCH1” denotes a receptor membrane protein encoded by NOTCH1 gene (Entrez Gene: 4851; Ensembl: ENSG00000148400; OMIM: 190198; UniProt: P46531). NOTCH1 gene encodes a class I transmembrane protein functioning as a ligand-activated transcription factor and playing an important role in cell differentiation, proliferation, and apoptosis. NOTCH1 undergoes multiple proteolytic cleavages that allow its intracellular portion (ICN) to translocate to the nucleus upon ligand-binding, thus leading to transcriptional activation of multiple target genes (Fabbri G et al., J Exp Med. 2011).

As used herein, the term “NOTCH1-driven” denotes a state wherein NOTCH1 pathway is activated, hyperactively or constitutively. For instance, NOTCH1 pathway can be activated by genetic alterations, ligand mediated or negative regulators alterations. In order to identify a hyper- or constitutive activation of a NOTCH1 pathway, the regulation of NOTCH1 target genes (as example HES1 and HEY1) and/or NOTCH1-ICN1 protein expression can be measured (Kluk et al., PLoS ONE, 2013). In particular, a significant upregulation of NOTCH1 target genes and/or a significant increasement of NOTCH1-ICN1 protein expression, as compared to a normal cell, indicates that NOTCH1 is activated. NOTCH1 mutational status can be identified with a DNA sequencing, PCR analysis or any genotyping method known in the art. Examples of such methods include, but are not limited to, chemical assays such as allele specific hybridation, primer extension, allele specific oligonucleotide ligation, sequencing, enzymatic cleavage, flap endonuclease discrimination; and detection methods such as fluorescence, chemiluminescence, and mass spectrometry.

In some embodiments, the NOTCH1 mutational status indicate a gain of function mutation. As used herein, the term “gain of function mutation” denotes any mutation in a gene in which the protein encoded by the gene (i.e., the mutant protein) acquires a function not normally associated with the protein (i.e., the wild-type protein). NOTCH1 gain-of-function mutations are well known in the art and can be a deletion, addition, or substitution of a nucleotide or nucleotides in the gene which gives rise to the change in the function of the encoded protein. Exemplary mutations are described in the literature (Weng et al., Science, 2004; O'Neil et al., Blood, 2006) and are encompassed in the invention. Mutations in NOTCH1 may be identified by any suitable method in the art, but in some embodiments the mutations are identified by one or more of polymerase chain reaction, sequencing, as well as immunostaining method using anti-mutant-NOTCH1 antibody.

Thus in some embodiments, the present invention relates to a method of treating a NOTCH1-driven cancer in a subject in need thereof comprising determining NOTCH1 mutational status and administering a ferroptosis inducer when the subject harbours a NOTCH1 gain of function mutation.

As used herein, the term “ferroptosis” denotes a regulated cell death program iron dependent characterized by the accumulation of lipid peroxides. Ferroptosis is initiated by elevated mitochondrial ROS production and failure in the oxidative stress response mediated by the GSH redox system, leading to the production of toxic peroxidised lipids (Dixon S. J. et al., Cell. 2013).

As used herein, the term “ferroptosis inducer” denotes a compound able to increase ferroptosis occurrence. Ferroptosis inducers are well-known in the art. As example, the ferroptosis inducer may be APR-246, Ras Synthetic Lethal 3 (RSL3), ML162, ML210, acrolein, erastin, Imidazole Ketone Erastin (IKE), Piperazine Erastin (PE), sulfasalazine, sorafenib, Ferroptosis Inducer 56 (FIN56), Ferroptosis inducer endoperoxide (FIN02), Caspase-Independent Lethal 56 (CIL56), mevalonate-derived coenzyme Q10, buthionine sulfoximine (BSO), amentoflavone, dihydroartemisinin (DHA), typhaneoside, artesunate, Withaferin A (WA), auranofin.

In some embodiments, the ferroptosis inducer is APR-246. APR-246, also named Eprenetapopt or PRIMA-1MET (CAS number: 5291-32-7) is a small organic molecule of formula (I). APR-246 is known to exerts a p53-independent function via the depletion of glutathione (GSH) and the accumulation of mitochondrial ROS, leading to the induction of ferroptosis.

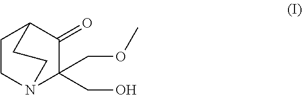

In some embodiments, the mutational status of the subject is TP53^{wt} NOTCH1^{mut}.
In some embodiments, the mutational status of the subject is TP53^{alt} NOTCH1^{mut}.

As used herein, the term “TP53” or “Tumor Protein 53” denotes a transcription factor encoded by TP53 gene (Entrez gene: 7157; Ensembl: ENSG00000141510; OMIM: 191170; UniProt: P04637). The term “TP53^{wt}” indicates that the expression of TP53 is not altered. The term “TP53^{alt}” indicated that the expression of TP53 is altered. In the context of the invention, the term “altered” refers to a state wherein a gene is mutated (i.e. addition or substitution) or deleted and/or wherein the protein expression is downregulated or eradicated. In some embodiments, TP53 gene alteration is induced by a missense mutation.

As used herein, the term “treatment” or “treat” refers to both prophylactic or preventive treatment as well as curative or disease modifying treatment, including treatment of subject at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the expression “therapeutically effective amount” is meant a sufficient amount of the active ingredient (e.g. ferroptosis inducer) for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiments, the ferroptosis inducer is administrated in combination with at least one anticancer agent.

As used herein, the term “combination” encompasses simultaneous, separate or sequential use of two therapeutic compounds.

Anti-cancer agents may be for example doxorubicin, etoposide (VP-16), azacytidine, venetoclax, acalabrutinib, blimatumomab, inotuzumab, rituximab, cytarabine, gemcitabine, tamoxifen, anthracyclines, fludarabine, capecitabine, methotrexate, taxol, taxotere, mercaptopurine, thioguanine, hydroxyurea, cyclophosphamide, ifosfamide, nitrosoureas, platinum complexes such as cisplatin, carboplatin and oxaliplatin, mitomycin, dacarbazine, procarbizine, teniposide, campathecins, bleomycin, idarubicin, epirubicin, daunorubicin, dactinomycin, plicamycin, mitoxantrone, L-asparaginase, epimbicm, 5-fluorouracil, taxanes such as docetaxel and paclitaxel, leucovorin, levamisole, irinotecan, estramustine, nitrogen mustards, BCNU, nitrosoureas such as carmustme and lomustine, vinca alkaloids such as vinblastine, vincristine and vinorelbine, imatimb mesylate, hexamethyhnelamine, topotecan, kinase inhibitors, phosphatase inhibitors, ATPase inhibitors, tyrphostins, protease inhibitors, inhibitors herbimycm A, genistein, erbstatin, and lavendustin A. In some embodiments, the ferroptosis inducer is APR-246 and the at least one anti-cancer agent is etoposide or doxorubicin.

Typically the active ingredient of the present invention (e.g. ferroptosis inducer) is combined with pharmaceutically acceptable excipients, and optionally sustained-release matrices, such as biodegradable polymers, to form pharmaceutical compositions. The term “pharmaceutical” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Thus, the invention also relates to a method of treating a NOTCH1-driven cancer in a subject in need thereof comprising administering a pharmaceutical composition comprising a ferroptosis inducer. In some embodiments, the ferroptosis inducer is APR-246. In some embodiments, the pharmaceutical composition comprises at least one anti-cancer agent. In some embodiments, the ferroptosis inducer is APR-246 and the at least one anti-cancer agent is etoposide or doxorubicin.

In some embodiments, the present invention relates to a method of treating a NOTCH1-driven cancer comprising determining NOTCH1 mutational status and administering a pharmaceutical composition comprising a ferroptosis inducer and at least one anti-cancer agent when the NOTCH1 mutational status indicate a gain of function mutation.

In a second aspect, the present invention relates to a method for predicting the response of a subject suffering from a NOTCH1-driven cancer to a ferroptosis inducer wherein a NOTCH1 pathway activation indicates that the subject is responder to the ferroptosis inducer.

In some embodiments, NOTCH1 pathway activation is determined by determining NOTCH1 mutational status.

In some embodiments, a NOTCH1 gain of function mutation indicate that the subject is responder to a ferroptosis inducer.

As used herein, the term “responder” denotes a state wherein the subject achieves a partial or complete response to a compound or composition. As example, a response is characterized when all of the cancer or tumor disappears, when the cancer has shrunk by percentage but disease remains or when the cancer has neither grown nor shrunk.

The invention will be further illustrated by the following figures and examples. However, these examples and figures should not be interpreted in any way as limiting the scope of the present invention.

### FIGURES

FIG. 1. Clinical impact of TP53 Alterations in the GRAALL0305 and FRALLE2000 studies. Overall survival (A) and cumulative incidence of relapse (B).

FIG. 2. APR-246 is effective and synergize with doxorubicine in T-ALL with TP53^{Mut}. (A) APR-246 induces cell death in TP53^{Mut} T-ALL cell lines. (B) APR-246 induces cell death in T-ALL TP53^{Mut} PDX. (C) APR-246 restores doxorubicin sensitivity in TP53^{Mut} T-cell lines. (D) Synergistic effects of APR-246 and doxorubicin in TP53^{Mut} T-ALL cell lines.

FIG. 3. T-ALL TP53^{WT} NOTCH1^{Mut} are sensitive to APR-246. (A) T-cell line TP53^{WT} NOTCH1^{Mut} are sensitive to APR-246. (B) T-ALL PDX TP53^{WT} NOTCH1^{Mut} are more sensitive to APR-246 than TP53^{WT} NOTCH1^{WT}. (C) Synergistic effects of APR-246 and VP-16 in T-ALL TP53^{WT} NOTCH1^{Mut} and TP53^{WT} NOTCH^{WT}PDX.

FIG. 4. CRISP/Cas9-induced NOTCH1-PEST truncation in T-ALL PDX TP53^{WT} NOTCH1^{WT}. (A) We introduce a frameshift mutation affecting the NOTCH1-PEST domain in a T-ALL PDX TP53^{WT} NOTCH1^{WT} (UPNT525) using CRISPR-cas9 system. (B) CRISP/Cas9-induced NOTCH1-PEST truncation leads to ICN1 accumulation in T-ALL PDX TP53^{WT} NOTCH1^{WT}. (C) Expression of Notch1 target genes (HES1 and HEY1) was elevated in CRISP/Cas9-induced NOTCH1-PEST T-ALL PDX. (D) CRISP/Cas9-induced NOTCH1-PEST truncation is associated with a higher sensitivity to APR-246.

FIG. 5. APR-246 induces Ferroptosis in T-ALL. (A,B) APR-246 induces ferroptosis (mitochondrial ROS, peroxidized lipids and GSH depletion) in both NOTCH1^{Mut} and NOTCH1^{WT} T-ALL PDX. (C) CRISPR/Cas9-induced NOTCH1-PEST truncation is associated with both increased basal and APR-246-induced level of ferroptosis.

### EXAMPLE

### Material and Methods

### Patients and Primary Samples

Adult patients were enrolled in the GRAALL-2003-2005 trials (GRAALL-2003, NCT00222027; GRAALL-2005, NCT00327678) and pediatric patients were enrolled in the FRALLE 2000 trial. Based on DNA availability for molecular analysis, 215 adult patients out of 337 and 261 pediatric patients out of 405 were included in this study. No difference in clinical outcomes was observed between the included patients and the entire cohort (data not shown). Diagnostic peripheral blood or bone marrow samples were collected after informed consent was obtained at diagnosis, according to the Declaration of Helsinki. All samples contained ≥80% blasts, immunophenotypic and molecular characterization of T-ALL samples, minimal residual disease (MRD) assessment, gene mutation screening, MLPA were performed as previously described (21, 26, 27).

### Next Generation Sequencing, Copy Number and Molecular Analysis

Genomic analysis was performed by pan-exon targeted next-generation sequencing (NGS) of DNA extracted from diagnostic samples; DNA libraries were prepared using Nextera XT kit (Illumina) and sequenced on an Illumina MiSeq. The NGS panel included 69 genes known to be mutated in T-ALL. NGS analysis was performed with Polyweb software (Imagine Institute). Genetic lesion co-occurrences and mutual exclusions were computed using the DISCOVER package.

Copy Number Evaluation from Next-Generation Sequencing (NGS) Data

We performed a computational approach previously described for the detection of copy number variants (CNVs) from next-generation sequence data (28), including a systematic analysis of depth of TP53 gene coverage. This is based on variations in depth of coverage of aligned sequence reads, using a locally developed algorithm. The CNVs detected were confirmed by high resolution CGH and/or MLPA analysis (kit P037-CLL-1 MRC-Holland).

### Microarray Based Comparative Genomic Hybridization (Array CGH)

Diagnostic DNA was hybridized on Affymetrix (Santa Clara, CA) Cytogenetics whole Genome 2.7M Arrays (CGH-array), according to the manufacturer's recommendations. Data analysis was performed with the Chromosome Analysis Suite (ChAS) software (Affymetrix®). Gene copy number (GCN) aberrations were compared with the Database of Genomic variants (DGV) http://projects.tcag.ca/variation to study only non-variant GCV aberrations.

### Cell Lines, Culture Conditions, and Treatments

The T-ALL cell lines Loucy, CEM, RPMI, PEER, MOLT-4 and SIL-ALL were grown in RPMI-1640 supplemented with 50 μg/mL streptomycin, 50 UI penicillin, 4 mM L-glutamine and 10% fetal bovine serum (complete medium). Cell lines were authenticated by the ATCC. For ex vivo experiments, PDX were cultured in complete medium, supplemented with 50 ng/ml human stem cell factor, 20 ng/ml hFLT3-L, 10 ng/ml hIL-7 and 20 nM insulin (MiltenyiBiotec). Cultures were maintained at 37° C. in a humidified atmosphere containing 5% CO₂. For treatment, cells or PDX were incubated with increasing doses of APR-246 during 48 hours. APR-246 was purchased from AbMole BioScience (Houston, USA). Doxorubicin and etoposide were obtained from Sigma-Aldrich (Saint-Louis, MO).

Generation of T-ALL PDX with NOTCH1-PEST Mutation Via CRISPR/Cas9 Technology

UNT525 NOTCH1-PEST mutation was constructed by the Alt-R CRISPR-Cas9 system (Integrated DNA Technologies, Inc.) Three different guide RNAs specific to NOTCH1 were screened and the oligo ACGTCGCTGCCATCCTCGCTG was chosen for further transfection experiments in UPNT525 PDX T-ALL. Indel mutations within the PEST domain were confirmed by NGS. hCD45+ T-ALL cells of UPNT525 PDX were electroporated with Cas9-gRNA ribonucleoprotein complexes for NOTCH1-PEST knock-in. gRNAs were synthesized from Integrated DNA Technologies as Alt-R CRISPR-Cas9 crRNA. The functional gRNA was created after annealing with Alt-R tracrRNA (Integrated DNA Technologies). Editing efficiency evaluated with TIDE algorithm (https://tide.nki.nl/) was around 75%.

### Flow Cytometry-Based Assay

Data acquisition and data analysis were conducted at the INEM institute. Cell viability was determined by flow cytometry by Annexin V-APC/propidium iodide co-staining (BD-Pharmingen, San Jose, CA, USA). Lipid peroxide production measurements was determined using C11-BODIPY (581/591) (2 μM, Thermo Fisher, Waltham, MA). Mitochondrial superoxide levels were measured by MitoSOX™ Red staining (M36008, Thermo Fisher, Waltham, MA). Glutathione measurements was determined using monochlorobimane (MCB; Thermo Fisher, Waltham, MA).

### Western Blotting

Cells were washed in Phosphate Buffered Saline (PBS) and lysed in RIPA buffer (20 mM Tris-HCl, pH 7.5, 150 mM NaCl, 1 mM EDTA, 1 mM EGTA, 1% NP-40, 1% sodium deoxycholate, 2.5 mM sodium pyrophosphate, 1 mM 0-glycerophosphate, 1 mM Na3VO4) supplemented with protease and phosphatase inhibitors (Halt Protease and Phosphatase Inhibitor Cocktail, Thermo Scientific). Cell lysates were separated by SDS-PAGE and transferred onto nitrocellulose membranes. After blocking 1 h in blocking buffer (Tween-PBS 5% bovine serum albumin), membranes were incubated overnight at 4° C. with the primary antibodies in blocking buffer (NOTCH1-ICN1). After washes, membranes were incubated for 1 h at room temperature with the appropriate secondary antibodies coupled to HRP in blocking buffer. Chemiluminescence signals were revealed using the WestDura Super Signal kit (GE Healthcare Bio-Sciences) on a ChemiDoc XRS from Bio-Rad.

### Measurement of Synergistic Effects

Cell viability was calculated for every dose combination of APR-246 and topoisomerase inhibitor (doxorubicin or etoposide) using the Synergy Finder webtool (https://synergyfinder.fimm.fi/) and compared to each agent alone during 48 h. Calculations were based on the ZIP model (29).

### Patient-Derived Xenografts

Patient-derived xenografts (PDXs) were generated from primary T-ALL samples as described (30). Briefly, 10⁶ viable leukemic cells were xenografted by intravenous retro-orbital injection in 2-month-old NSG mice. Mice were monitored weekly by flow cytometry for leukemic load (FSChi, hCD7+, hCD45+ cells) in peripheral blood. Mice were euthanized when terminally ill, as evidenced by either severe dyspnea or weakness caused by leukemic dissemination in the thymus or vital organs. Bone marrows from tibiae, hip, femora and vertebrae were collected for subsequent ex vivo experiments. All samples used contained ≥90% blasts.

### Statistical Analysis

Comparisons for categorical and continuous variables between TP53^{Mut} and TP53^{WT} subgroups were performed with Fisher's exact test and Mann-Whitney test respectively. Normal distributions were verified prior to conducting parametric tests. Overall survival (OS) was calculated from the date of diagnosis to the last follow-up date censoring living patients. The cumulative incidence of relapse (CIR) was calculated from the complete remission date to the date of relapse censoring patients alive without relapse at the last follow-up date. Relapse and death in complete remission were considered as competitive events. Univariate and multivariate analyses assessing the impact of categorical and continuous variables were performed with a Cox model. Proportional-hazards assumption was checked before conducting multivariate analyses. In univariate and multivariate analyses, age and log₁₀(WBC) were considered as continuous variables. All analyses were stratified on the trial. Variables with a p-value less than 0.1 in univariate analysis were included in the multivariable models. Statistical analyses were performed with STATA software (STATA 12.0 Corporation, College Station, TX). All p-values were two-sided, with p<0.05 denoting statistical significance. The Circos plot and the Oncoplot were generated using R software.

### Study Approval

GRAALL0305 and FRALLE2000 trials were conducted in accordance with the Declaration of Helsinki and approved by local and multicenter research ethical committees. The experimental procedures were approved by the Institute Animal Care Committee.

### Results

### Oncogenetic Landscape and Clinical Impact of TP53 Alterations in T-ALL

We investigated the clinical characteristics linked to TP53^{Alt} in 476 patients, including 251 adults enrolled in the GRAALL-2003/2005 trials, and 261 children enrolled in the FRALLE-2000 trial (Table 1). The incidence of TP53^{Alt} at diagnosis in these cohorts was 4% (21/476). TP53^{Mut} were detected in 9 patients (6 adults and 3 pediatric cases, data not shown) and TP53^{Del} were identified in 15 patients (7 adults and 8 pediatric cases), 3 patients harbored both TP53^{Mut} and TP53^{Del}. Patients with TP53^{Alt} did not significantly differ from TP53^{Wt} patient regarding sex, age and central nervous system (CNS) involvement (Table 1), but were associated with an immature phenotype (8/20, 40% vs 81/399, 20%, p=0.048). The oncogenetic landscape of TP53^{Alt} was comparable to TP53 wild-type (TP53^{Wt}) T-ALLs (data not shown).

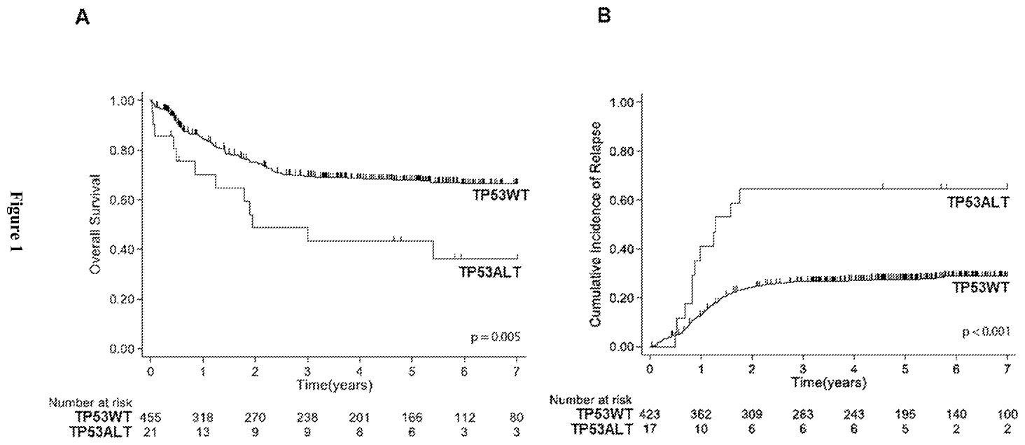

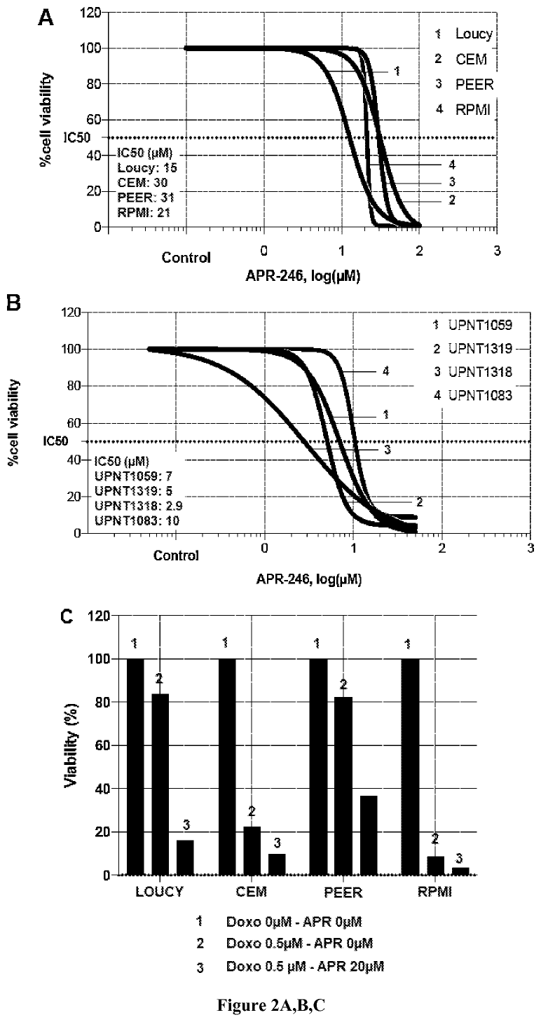

To investigate the prognostic value of TP53^{Alt}, survival analyses were performed on the series of 476 patients. TP53^{Alt} did not confer increased poor prednisone response, defined by a peripheral blood blast count >1.0×10⁹/L at the end of the induction phase) (38% vs 45%, p=0.7) (Table 1). Although TP53Alt did not significantly influence the morphological complete response rate at the end of induction course (81% vs 93%, p=0.07), patients harboring TP53^{Alt} were associated with delayed early medullary blast clearance, as confirmed by end of induction MRD1 assessment, with more positivity (≥10-4) in TP53^{Alt} as compared with TP53^{Wt} cases (75% vs 35%, p=0.01). Patients with TP53^{Alt} had an inferior outcome compared to TP53^{Wt} (Table 1, FIGS. 1A and 1B) with an increased cumulative incidence of relapse (CIR) (5y-CIR: 65% vs. 27%; specific hazard ratio (SHR) 3.1, 95% CI (1.67-5.78); p<0.001) and a shorter overall survival (OS) (5y-OS: 48% vs. 72%; hazard ratio: 2.34, 95% CI (1.30-4.24); p=0.005). In multivariate analysis TP53^{Alt} predicted a statistically lower OS (HR: 2.87, 95% CI (1.56-5.26); p=0.001) and higher CIR (SHR, 2.90, 95% CI 1.55-5.44), p=0.001) even after adjustment on the 4-genes NOTCH1/FBXW7/RAS/PTEN (NFRP) classifier, which identified poor prognosis patients in both GRAALL and FRALLE trials (21, 22).

This study provides the largest comprehensive analysis of TP53^{Alt} in T-ALL, describing for the first time both their clinical profile and, most importantly, the extremely poor prognosis impact associated with TP53^{Alt} at diagnosis in T-ALL, urging the need to develop innovative targeted therapies for patients harboring TP53^{Alt} at diagnosis and/or relapse.

T-ALL with TP53^{Mut} are Sensitive to APR-246

Negative outcomes observed in T-ALL harboring TP53^{Alt} are likely to be related to p53-induced therapeutic resistance previously described for other malignancies with p53 mutations (23, 24), including the loss of wild-type p53 tumor suppressor activity and acquisition of novel functions that disrupt the DNA-damage response pathway and permit tumor survival upon oncogenic stress. APR-246 has recently shown promising results in TP53^{Mut} acute myeloid leukemia and myelodysplastic syndromes (25). As TP53^{Mut} are strong drivers of negative outcomes in T-ALL, restoring wild-type function in TP53 mutated cases could have beneficial impact. We therefore evaluated APR-246 sensitivity in TP53^{Mut} T-ALL.

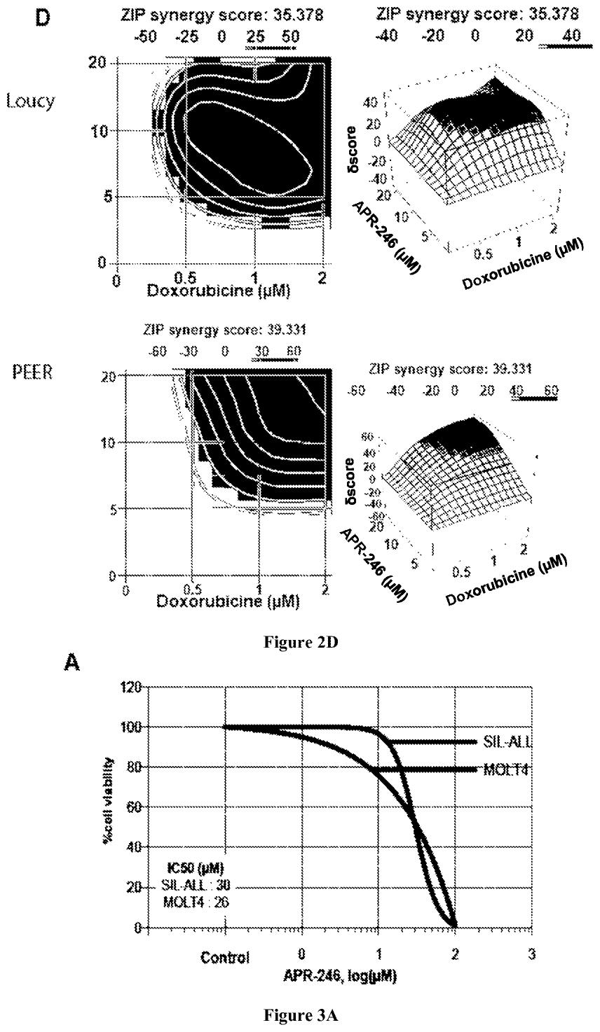

We characterized four T-ALL cell lines with previously described missense TP53^{Mut}; CEM, RPMI-8402, LOUCY and PEER (p53.iarc.fr and data not shown). As expected, all 4 were sensitive to APR-246 (FIG. 2A). Similar results were obtained from four patient-derived T-ALL TP53^{Mut} samples at diagnosis (UPNT1059 and UPNT1083) or at relapse (UPNT1318 and UPNT1319) established in our NOD/SCID/huALL patient derived xenografts (PDX) (FIG. 2B and data not shown), confirming that APR-246 has strong cytotoxic activity in TP53^{Mut} T-ALL. Interestingly, a derived primograft sample (UPNT1318) harboring an acquired-TP53^{Mut} at relapse showed higher sensitivity to APR-246 than the TP53^{WT} diagnosis-matched sample (data not shown). We confirmed that APR-246 reactivated the p53 pathway and resulted in cell death by promoting apoptosis in TP53^{Mut} T-ALL (data not shown) compared to TP53^{WT} T-ALL (data not shown).

TP53^{Mut} cancer cells are associated with DNA-damage resistance. Hence, we analyzed whether APR-246 could restore the sensitivity to the DNA-damaging agent doxorubicin (a common treatment of T-ALL) via the re-conformation of mutant p53 in a wild-type scaffold.

As expected, T-cell lines harboring TP53^{Mut} showed relative resistance to doxorubicin. Importantly, the combination therapy of APR-246 and doxorubicin presented a strong synergy in T-ALL cell lines TP53^{Mut} (FIGS. 2C and 2D). These results suggest that the addition of APR-246 to an existing p53-dependent chemotherapy could be an efficient strategy to target TP53^{Mut} T-ALL and overcome the chemoresistance in these patients.

### Notch1 and p53 Interplay Primes T-ALL to APR-246 Sensitivity

Recent studies shed light on the complex interplay between Notch1 and p53 signaling pathways. Critically, Notch1 acts as a repressor of p53 function through diverse mechanisms including a direct protein-protein interaction between the N-terminal domain of Notch1 and p53 resulting in the inhibition of p53 phosphorylation and DNA binding activity. This crosstalk between Notch1 and p53 may constitute a main driver for leukemia onset and progression. Hence, we hypothesized that APR-246 could restore p53^{WT} function and exert anti-leukemic activity in Notch1-altered TP53^{WT}-ALL.

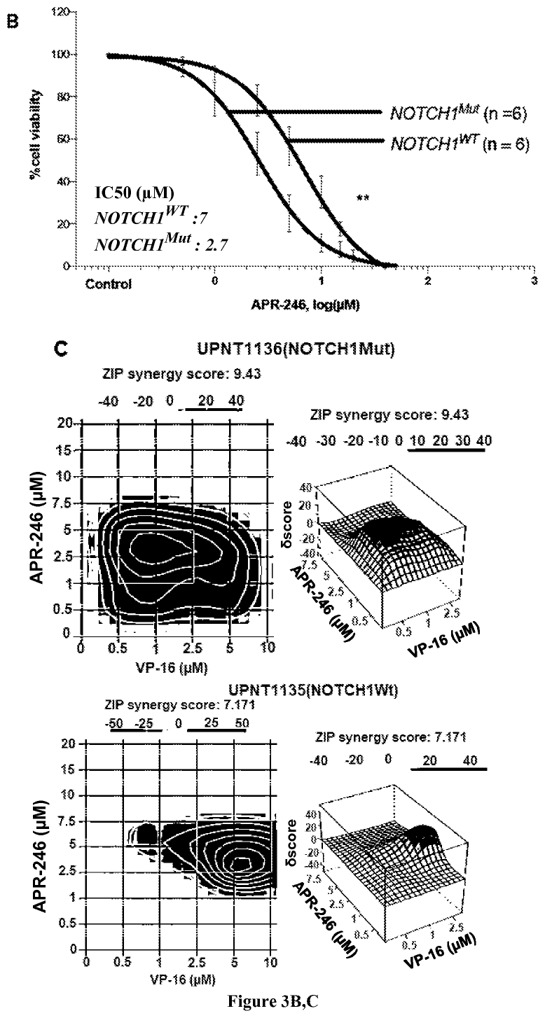

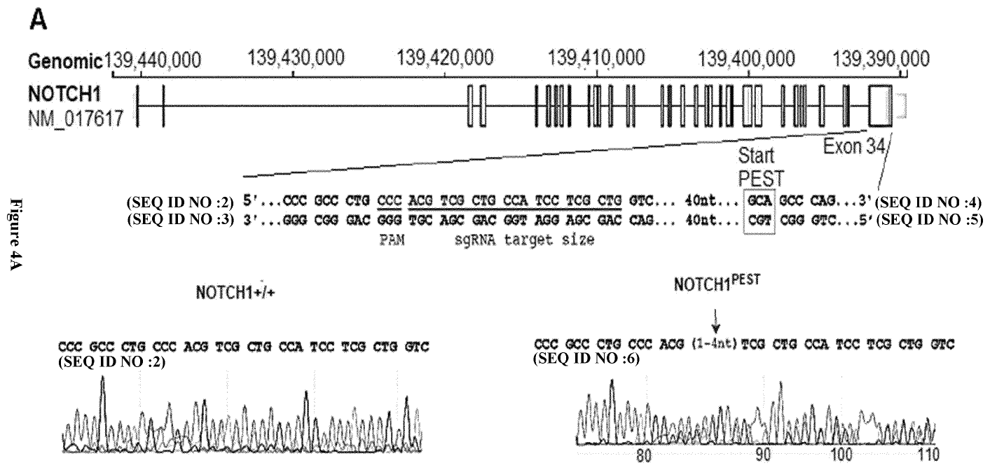

We characterized two TP53^{WT} NOTCH1^{Mut} T-ALL cell lines (MOLT-4 and SIL-ALL) and identified that APR-246 significantly altered cell survival in these NOTCH1^{Mut} TP53^{WT} T-ALL cell lines (FIG. 3A). Importantly, similar results were obtained with 6 TP53^{WT} NOTCH1^{Mut} T-ALL PDX (FIG. 3B). Compared to TP53WT NOTCH1Mut, T-ALL patients samples TP53^{WT} NOTCH1^{WT} were significantly less sensitive to APR-246. (FIG. 3B). Importantly, while APR-246 showed a synergistic effect with the VP-16 topo-isomerase II inhibitor, irrespective of NOTCH1 mutational status, NOTCH1^{Mut} T-ALL PDX exhibited a higher sensitivity profile than NOTCH1^{WT} PDX (FIG. 3C). Altogether, these data suggest that Notch1 activation could regulate APR-246 efficacy in T-ALL.

To functionally demonstrate the relationship between Notch1 activation and cellular sensitivity to APR-246, we first co-cultured the Loucy T-ALL cell line Loucy (TP53^{WT} NOTCH1^{Mut}) on OP9-DL1, thus activating the Notch1 pathway via Notch1 Delta-like ligand 1 (DL-1) interaction. This led to Notch1 pathway activation, as confirmed by both the increase of Notch1-ICN1 protein expression and the upregulation of Notch1 target genes (data not shown). Critically, Loucy exhibited higher sensitivity to APR-246 when co-cultured on OP9-DL1, reinforcing the interplay between Notch1 signaling and AP-246 efficacy (data not shown).

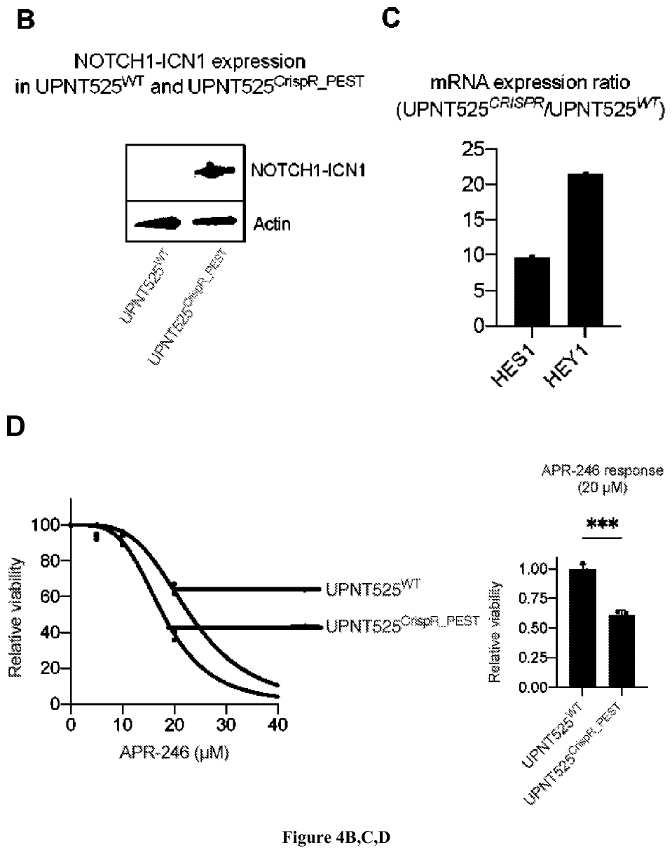

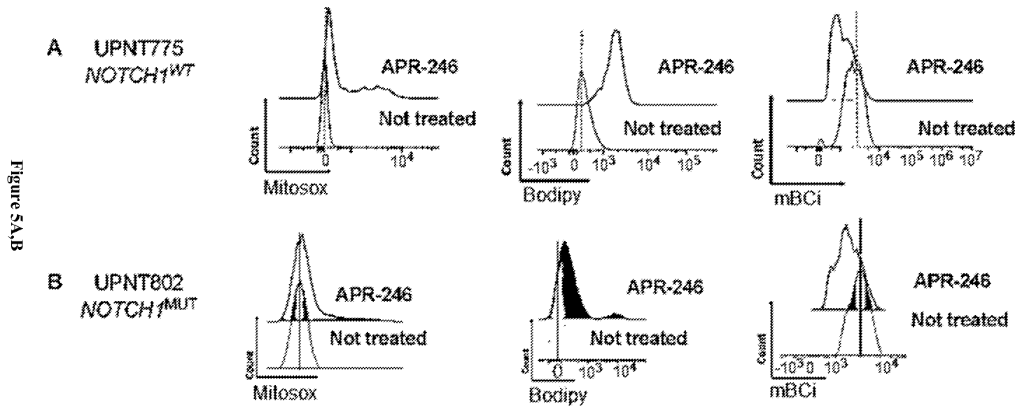

Additionally, we introduced a NOTCH1-PEST mutation using CRISPR/Cas9 in a TP53^{WT}NOTCH1^{WT} T-ALL PDX (UPNT-525) (FIG. 4A). As expected, this increased Notch1-ICN1 protein expression and subsequent Notch1 pathway target genes expression (mRNA HES1 and HEY1) (FIGS. 4B and 4C). While the parental PDX is relatively resistant, activation of the Notch1 pathway efficiently sensitizes to APR-246 (FIG. 4D). These results strongly suggest that aberrant Notch1 signaling regulates APR-246 sensitivity by conferring vulnerability to p53 restoration, even in a p53-proficient background. The presence of NOTCH1 mutations could therefore represent a predictive biomarker for the response to APR-246.

### Notch1 Activation Primes T-ALL to Ferroptosis

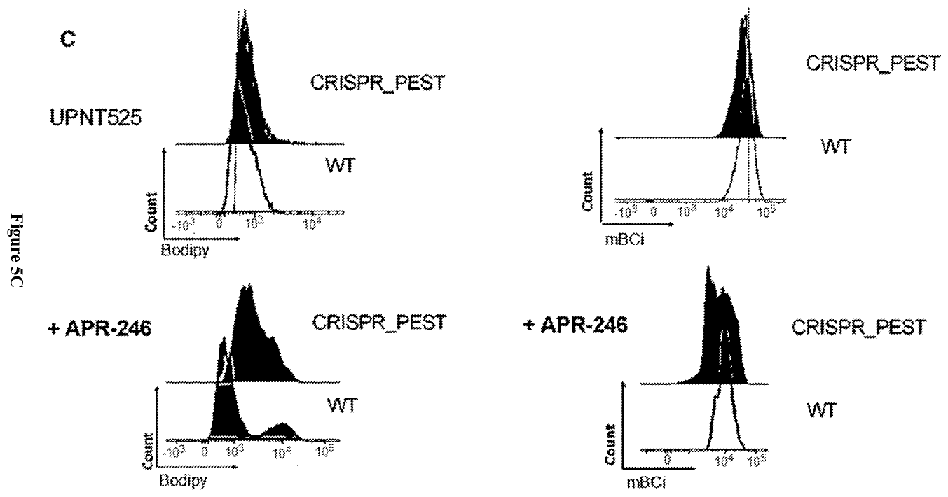

We further sought to address the mode of action of APR-246 in TP53^{WT} T-ALL and the mechanisms by which NOTCH1^{Mut} sensitizes to APR-246. Since APR-246 exerts a p53-independent function via the depletion of glutathione (GSH) and the accumulation of mitochondrial ROS, leading to the induction of ferroptosis (20,21), we examined whether the anti-leukemic activity of APR-246 was mediated by ferroptosis in T-ALL. This alternative cell death mechanism is characterized by elevated mitochondrial ROS production and failure of the oxidative stress response mediated by the GSH redox system, leading to production of toxic peroxidized lipids. Ex vivo APR-246 treatment induced a net increase in mitochondrial ROS, marked depletion in reduced glutathione levels (GSH), and an accumulation of peroxidized lipids in TP53WT NOTCH1^{WT/Mut} (FIGS. 5A and 5B) and in TP53^{Mut} T-ALL (data not shown), reinforcing the concept that APR-246 induces ferroptosis irrespective of TP53 mutational status. Of note, these ferroptosis markers were partially reversed by ferrostatin-1 treatment, a lipophilic antioxidant that delays lipid peroxidation, thus confirming the induction of ferroptosis (data not shown).

To explore the mechanism underlying the high sensitivity to APR-246 of TP53^{WT}NOTCH1^{Mut} T-ALL compared to TP53^{WT} NOTCH1^{WT}, we evaluated the basal levels of ferroptosis in our two inducible Notch1 pathways models in absence and after exposition to APR-246. Critically, Notch1 activation enhanced the level of lipid peroxidation and the GSH depletion (FIG. 5C and data not shown) in both conditions (with and without APR-246).

These results confirm that the Notch1/p53 interplay could modulate the basal level of ferroptosis and could explain the subsequent responsiveness to APR-246 in TP53^{WT} NOTCH1^{Mut} T-ALL. Overall, our data provide a strong rationale for the use of p53-targeting APR-246 in NOTCH1-driven malignancies.

### CONCLUSION

We propose that Notch1 activation conveys a favorable context eliciting APR-246 sensitivity. Hence, the activation of Notch1 signaling predicts the response to APR-246. Also, this study provides a strong rationale for targeting p53 in T-ALL and, more importantly, suggests a potent efficacy of APR-246 on Notch1-altered T-ALL, which represents 70% of the T-ALL. Our results shed light on Notch1-driven vulnerabilities that can be exploited by targeted therapies such as APR-246 and provide a rationale for its clinical use beyond the scope of leukemia.

### TABLES

TABLE 1
TP53 Alterations in T-ALL (FRALLE and GRAALL patients)

| | TP53^{Alt} | | TP53^{WT} | | Total | | |
|---|---|---|---|---|---|---|---|
| Variable | n = 21 (4%) | | n = 455 (96%) | | n = 476 | | p-value² |
| Male | 14/21 | (67%) | 343/455 | (75%) | 357/476 | (75%) | 0.4 |
| Age (y)¹ | 23.4 | (4.0-51.8) | 15.3 | (1.1-59.1) | 15.3 | (1.1-59.1) | 0.5 |
| WBC (G/I)¹ | 25 | (5-674) | 66 | (0-980) | 64 | (0-980) | 0.01 |
| CNS Involvement | 3/21 | (14%) | 48/453 | (11%) | 51/474 | (11%) | 0.5 |

Immunophenotype

| | | | | | | | |
|---|---|---|---|---|---|---|---|
| ETP phenotype | 6/16 | (38%) | 50/291 | (17%) | 56/307 | (18%) | 0.09 |
| Immature (IM0/δ/γ) | 8/20 | (40%) | 81/399 | (20%) | 89/419 | (50%) | 0.048 |
| αβ lineage | 6/20 | (30%) | 205/399 | (51%) | 211/419 | (50%) | 0.07 |
| Mature TCRγδ | 3/20 | (15%) | 63/399 | (16%) | 66/419 | (16%) | >0.9 |

Oncogenetic classification

| | | | | | | | |
|---|---|---|---|---|---|---|---|
| TLX1 | 1/18 | (6%) | 53/397 | (13%) | 54/415 | (13%) | 0.5 |
| TLX3 | 2/18 | (11%) | 70/397 | (18%) | 72/415 | (17%) | 0.8 |
| SIL-TAL1 | 1/18 | (6%) | 56/397 | (14%) | 57/415 | (14%) | 0.5 |
| CALM-AF10 | 0/18 | (0%) | 13/397 | (3%) | 13/415 | (3%) | >0.9 |
| High-Risk Classifier | 13/21 | (62%) | 196/455 | (43%) | 209/476 | (44%) | 0.12 |

Treatment Response

| | | | | | | | |
|---|---|---|---|---|---|---|---|
| Prednisone response | 13/21 | (62%) | 246/446 | (55%) | 259/467 | (55%) | 0.7 |
| Chemosensitivity | 12/21 | (57%) | 325/446 | (73%) | 337/467 | (72%) | 0.1 |
| MRD1 > 10⁻⁴ | 9/12 | (75%) | 114/328 | (35%) | 123/340 | (36%) | 0.01 |
| Complete Remission | 17/21 | (81%) | 423/455 | (93%) | 440/476 | (92%) | 0.07 |
| Allo-HSCT | 4/20 | (20%) | 97/436 | (22%) | 101/456 | (22%) | 0.7 |

Outcome

| | | | | | | | |
|---|---|---|---|---|---|---|---|
| 5-year CIR [95% CI] | 65% | [11; 43] | 27% | [23; 32] | 29% | [25; 33] | <0.001 |
| 5-year OS [95% CI] | 48% | [26; 67] | 72% | [68; 76] | 71% | [67; 75] | 0.005 |

T-ALL: T-cell acute lymphoblastic leukemia; WBC, white blood count; CNS, central nervous system; ETP, early thymic precursor; High Risk Classifier, NOTCH1/FBXW7-RAS/PTEN classifier as previously described; CR, complete remission; MRD, minimal residual disease; Allo-HSCT, allogenic hematopoietic stem cell transplantation; CIR, cumulative incidence of relapse; OS, overall survival; HR: hazard ratio, SHR: specific hazard ratio, CI: confidence interval

| | Univariate and Multivariate Analysis³ | | | | | |
|---|---|---|---|---|---|---|
| | Univariate | | | Multivariate | | |
| CIR | SHR | 95% CI | p | SHR | 95% CI | p |
| Age | 1.01 | (0.98; 1.03) | 0.57 | — | — | — |
| CNS | 1.57 | (0.85; 2.59) | 0.08 | 1.28 | (0.77; 2.13) | 0.34 |
| Log(WBC) | 1.62 | (1.2; 2.18) | 0.002 | 1.62 | (1.19; 2.19) | 0.002 |
| Prednisone response | 0.67 | (0.47; 0.95) | 0.03 | 0.93 | (0.64; 1.35) | 0.70 |
| High-risk Classifier | 2.78 | (1.94; 3.99) | <0.001 | 2.58 | (1.78; 3.74) | <0.001 |
| TP53^{Alt} | 3.11 | (1.67; 5.78) | <0.001 | 2.90 | (1.55; 5.44) | 0.001 |
| OS | HR | 95% CI | p | HR | 95% CI | p |
| Age | 1.03 | (1.01; 1.05) | 0.001 | 1.05 | (1.03; 1.07) | <0.001 |
| CNS | 2.00 | (1.28; 3.14) | 0.002 | 1.64 | (1.02; 2.64) | 0.04 |
| Log (WBC) | 1.99 | 1.48; 2.67) | <0.001 | 2.01 | (1.51; 2.86) | <0.001 |
| Prednisone response | 0.54 | (0.38; 0.76) | <0.001 | 0.83 | (0.57; 1.20) | 0.31 |
| High-risk Classifier | 2.93 | (2.06; 4.17) | <0.001 | 2.90 | (2.01; 4.18) | <0.001 |
| TP53^{Alt} | 2.34 | (1.30; 4.24) | 0.005 | 2.87 | (1.56; 5.26) | 0.001 |

¹ Statistics presented: Median (Minimum-Maximum)
² Statistical tests performed: Fisher's exact test; Wilcoxon rank-sum test.
³Univariate and multivariate Cox analyses stratified on protocol
p-values < 0.05 are indicated in bold
MRD1 correspond to MRD evaluation after induction and was performed by allele-specific oligonucleotides polymerase chain reaction. T-cell receptor status and oncogenic were performed as described in supplemental methods.

### REFERENCES

Throughout this application, various references describe the state of the art to which this invention pertains. The disclosures of these references are hereby incorporated by reference into the present disclosure.
1. Desjonqueres A et al. Acute lymphoblastic leukemia relapsing after first-line pediatric-inspired therapy: a retrospective GRAALL study. Blood Cancer J 2016; 6(12):e504.
2. Bhojwani D, Pui C-H. Relapsed childhood acute lymphoblastic leukaemia. Lancet Oncol 2013; 14(6):e205-217.
3. Levine A J. p53: 800 million years of evolution and 40 years of discovery. Nature Reviews Cancer 2020; 20(8):471-480.
4. Kawamura M et al. Alterations of the p53, p21, p16, p15 and RAS genes in childhood T-cell acute lymphoblastic leukemia. Leuk Res 1999; 23(2):115-126.
5. Van Vlierberghe P et al. Prognostic relevance of integrated genetic profiling in adult T-cell acute lymphoblastic leukemia. Blood 2013; 122(1):74-82.
6. Richter-Pechánska P et al. Identification of a genetically defined ultra-high-risk group in relapsed pediatric T-lymphoblastic leukemia. Blood Cancer Journal 2017; 7(2):e523.
7. Hof J et al. NOTCH1 mutation, TP53 alteration and myeloid antigen expression predict outcome heterogeneity in children with first relapse of T-cell acute lymphoblastic leukemia. Haematologica 2017; 102(7):e249-e252.
8. Diccianni M B et al. Clinical significance of p53 mutations in relapsed T-cell acute lymphoblastic leukemia. Blood 1994; 84(9):3105-3112.
9. Weng A P et al. Activating mutations of NOTCH1 in human T cell acute lymphoblastic leukemia. Science 2004; 306(5694):269-271.
10. Dotto G P. Crosstalk of Notch with p53 and p63 in cancer growth control. Nat Rev Cancer 2009; 9(8):587-595.
11. Kim S B et al. Activated Notch1 interacts with p53 to inhibit its phosphorylation and transactivation. Cell Death & Differentiation 2007; 14(5):982-991.
12. Genescà E et al. Frequency and clinical impact of CDKN2A/ARF/CDKN2B gene deletions as assessed by in-depth genetic analyses in adult T cell acute lymphoblastic leukemia [Internet]. J Hematol Oncol 2018; 11. doi:10.1186/s13045-018-0639-8
13. Beverly L J, Felsher D W, Capobianco A J. Suppression of p53 by Notch in lymphomagenesis: implications for initiation and regression. Cancer Res 2005; 65(16):7159-7168.
14. Zhou B P et al. HER-2/neu induces p53 ubiquitination via Akt-mediated MDM2 phosphorylation. Nature Cell Biology 2001; 3(11):973-982.
15. Mayo L D, Dixon J E, Durden D L, Tonks N K, Donner D B. PTEN protects p53 from Mdm2 and sensitizes cancer cells to chemotherapy. J Biol Chem 2002; 277(7):5484-5489.
16. Hasegawa H et al. Activation of p53 by Nutlin-3a, an antagonist of MDM2, induces apoptosis and cellular senescence in adult T-cell leukemia cells. Leukemia 2009; 23(11):2090-2101.
17. Lambert J M R et al. PRIMA-1 reactivates mutant p53 by covalent binding to the core domain. Cancer Cell 2009; 15(5):376-388.
18. Bykov V J N et al. Restoration of the tumor suppressor function to mutant p53 by a low-molecular-weight compound. Nat Med 2002; 8(3):282-288.
19. Liu D S et al. Inhibiting the system xC-/glutathione axis selectively targets cancers with mutant-p53 accumulation. Nat Commun 2017; 8:14844.
20. Birsen R et al. APR-246 induces early cell death by ferroptosis in acute myeloid leukemia. Haematologica [published online ahead of print: Jan. 7, 2021]; doi:10.3324/haematol.2020.259531
21. Trinquand A et al. Toward a NOTCH1/FBXW7/RAS/PTEN-based oncogenetic risk classification of adult T-cell acute lymphoblastic leukemia: a Group for Research in Adult Acute Lymphoblastic Leukemia study. J. Clin. Oncol. 2013; 31(34):4333-4342.
22. Petit A et al. Oncogenetic mutations combined with MRD improve outcome prediction in pediatric T-cell acute lymphoblastic leukemia. Blood 2018; 131(3):289-300.
23. Hientz K, Mohr A, Bhakta-Guha D, Efferth T. The role of p53 in cancer drug resistance and targeted chemotherapy. Oncotarget 2016; 8(5):8921-8946.
24. Mantovani F, Collavin L, Del Sal G. Mutant p53 as a guardian of the cancer cell. Cell Death & Differentiation 2019; 26(2):199-212.
25. Cluzeau T et al. Eprenetapopt Plus Azacitidine in TP53-Mutated Myelodysplastic Syndromes and Acute Myeloid Leukemia: A Phase II Study by the Groupe Francophone des Myélodysplasies (GFM). J Clin Oncol 2021; JCO2002342.
26. Bond J et al. An early thymic precursor phenotype predicts outcome exclusively in HOXA-overexpressing adult T-cell acute lymphoblastic leukemia: a Group for Research in Adult Acute Lymphoblastic Leukemia study. Haematologica 2016; 101(6):732-740.
27. Asnafi V et al. Analysis of TCR, pT alpha, and RAG-1 in T-acute lymphoblastic leukemias improves understanding of early human T-lymphoid lineage commitment. Blood 2003; 101(7):2693-2703.
28. Yoon S, Xuan Z, Makarov V, Ye K, Sebat J. Sensitive and accurate detection of copy number variants using read depth of coverage. Genome Res. 2009; 19(9):1586-1592.
29. Ianevski A, Giri A K, Aittokallio T. SynergyFinder 2.0: visual analytics of multi-drug combination synergies. Nucleic Acids Research 2020; 48(W1):W488-W493.
30. Trinquand A et al. Triggering the TCR Developmental Checkpoint Activates a Therapeutically Targetable Tumor Suppressive Pathway in T-cell Leukemia. Cancer Discov 2016; 6(9):972-985.

1. A method of treating a NOTCH1-driven cancer in a subject in need thereof comprising administering to the subject a therapeutically effective amount of a ferroptosis inducer.

2. The method of claim 1, further comprising determining a NOTCH1 mutational status of the subject and administering the ferroptosis inducer when the subject harbours a NOTCH1 gain of function mutation.

3. The method of claim 1, wherein the ferroptosis inducer is APR-246.

4. The method of claim 1, wherein the ferroptosis inducer is administrated in combination with at least one anticancer agent.

5. The method of claim 4, wherein the at least one anti-cancer agent is etoposide or doxorubicin.

6. The method of claim 1, wherein the NOTCH1-driven cancer is a T-cell acute lymphoblastic leukemia.

7. The method of claim 2, wherein the NOTCH1 mutational status of the subject is TP53^{wt}NOTCH1^{mut}.

8. The method of claim 2, wherein the NOTCH1 mutational status of the subject is TP53^{alt}NOTCH1^{mut}.

9. The method of claim 8, wherein the TP53^{alt}NOTCH1^{mut} is induced by a missense mutation.

10. A method for predicting the response of a subject suffering from a NOTCH1-driven cancer to a ferroptosis inducer wherein a NOTCH1 pathway activation indicates that the subject is a responder to the ferroptosis inducer.
